(19) United States
(12) Patent Application Publication
(10) Pub. No.: US 2017/0080129 A1
(43) Pub. Date: Mar. 23, 2017

(54) BIOLOGICAL MATERIAL-COATED DEVICES AND METHODS OF PRODUCING THE SAME

(71) Applicant: Ghassan S. Kassab, La Jolla, CA (US)

(72) Inventor: Ghassan S. Kassab, La Jolla, CA (US)

(21) Appl. No.: 15/268,986

(22) Filed: Sep. 19, 2016

Related U.S. Application Data
(60) Provisional application No. 62/219,902, filed on Sep. 17, 2015.

(51) Int. Cl. A61L 31/08; A61L 31/00; A61L 31/14

(52) U.S. Cl. CPC A61L 31/08; A61L 31/14; A61L 31/005; A61L 2420/02

(57) ABSTRACT

Biological material-coated devices and methods of producing the same. In a method to produce a coated medical device, the method comprises the steps of harvesting a tissue from a mammal, the tissue selected from the group consisting of pulmonary ligament, mediastinal pleura, parietal pleura, and visceral pleura, to obtain a harvested tissue; freezing the harvested tissue to obtain frozen tissue; mechanically processing the frozen tissue to obtain a liquid tissue product; and applying at least part of the liquid tissue product to at least part of a medical device so to at least partially coat the medical device with the liquid tissue, to produce a coated medical device.

### PRIORITY

The present application is related to, and claims the priority benefit of, U.S. Provisional Patent Application Ser. No. 62/219,902, filed Sep. 17, 2015, the contents of which are incorporated into the present disclosure in their entirety.

### BACKGROUND

Various medical devices, such as those which are temporarily or permanently positioned within the bloodstream, generally have the propensity for accumulating particulates found within the bloodstream, such as blood cells, cholesterol, etc., resulting in thrombi which can block blood flow and cause serious injury or even death of they are dislodged and trapped elsewhere within the body.

In view of the same, devices which perform as intended but reduce or eliminate bloodstream particulate accumulation would solve the problem noted above and would be well received in the marketplace.

### BRIEF DESCRIPTION

The present disclosure includes disclosure of a method to produce a coated medical device, the method comprising the steps of: harvesting a tissue from a mammal, the tissue selected from the group consisting of pulmonary ligament, mediastinal pleura, parietal pleura, and visceral pleura, to obtain a harvested tissue; freezing the harvested tissue to obtain frozen tissue; mechanically processing the frozen tissue to obtain a liquid tissue product; and applying at least part of the liquid tissue product to at least part of a medical device so to at least partially coat the medical device with the liquid tissue, to produce a coated medical device.

The present disclosure includes disclosure of a method to produce a coated medical device, wherein the tissue comprises pulmonary ligament, and wherein the step of mechanically processing the frozen tissue is performed by blending or grinding the frozen tissue.

The present disclosure includes disclosure of a method to produce a coated medical device, wherein the tissue comprises mediastinal pleura, and wherein the step of mechanically processing the frozen tissue is performed by blending or grinding the frozen tissue.

The present disclosure includes disclosure of a method to produce a coated medical device, wherein the tissue comprises parietal pleura, and wherein the step of mechanically processing the frozen tissue is performed by blending or grinding the frozen tissue.

The present disclosure includes disclosure of a method to produce a coated medical device, wherein the tissue comprises visceral pleura, and wherein the step of mechanically processing the frozen tissue is performed by blending or grinding the frozen tissue.

The present disclosure includes disclosure of a method to produce a coated medical device, wherein the step of applying is performed to apply at least part of the liquid tissue product to at least part of the medical device selected from the group consisting of a stent, a stent valve, an endoprosthesis, a catheter, a thrombus diverter, a lead, and a cannula.

The present disclosure includes disclosure of a method to produce a coated medical device, further comprising the step of combining a biologically-compatible adhesive with the liquid tissue product prior to the step of applying at least part of the liquid tissue product to at least part of the medical device.

The present disclosure includes disclosure of a method to produce a coated medical device, further comprising the step of applying a biologically-compatible adhesive to at least part of the medical device prior to the step of applying at least part of the liquid tissue product to at least part of the medical device.

The present disclosure includes disclosure of a coated medical device, generated by harvesting a tissue from a mammal, the tissue selected from the group consisting of pulmonary ligament, mediastinal pleura, parietal pleura, and visceral pleura, to obtain a harvested tissue; freezing the harvested tissue to obtain frozen tissue; mechanically processing the frozen tissue to obtain a liquid tissue product; and applying at least part of the liquid tissue product to at least part of a medical device so to at least partially coat the medical device with the liquid tissue, to produce a coated medical device.

The present disclosure includes disclosure of a coated medical device, wherein the tissue comprises pulmonary ligament, and wherein the step of mechanically processing the frozen tissue is performed by blending or grinding the frozen tissue.

The present disclosure includes disclosure of a coated medical device, wherein the tissue comprises mediastinal pleura, and wherein the step of mechanically processing the frozen tissue is performed by blending or grinding the frozen tissue.

The present disclosure includes disclosure of a coated medical device, wherein the tissue comprises parietal pleura, and wherein the step of mechanically processing the frozen tissue is performed by blending or grinding the frozen tissue.

The present disclosure includes disclosure of a coated medical device, wherein the tissue comprises visceral pleura, and wherein the step of mechanically processing the frozen tissue is performed by blending or grinding the frozen tissue.

The present disclosure includes disclosure of a coated medical device, wherein the step of applying is performed to apply at least part of the liquid tissue product to at least part of the medical device selected from the group consisting of a stent, a stent valve, an endoprosthesis, a catheter, a thrombus diverter, a lead, and a cannula.

The present disclosure includes disclosure of a coated medical device, further generated by combining a biologically-compatible adhesive with the liquid tissue product prior to the step of applying at least part of the liquid tissue product to at least part of the medical device.

The present disclosure includes disclosure of a coated medical device, further generated by applying a biologically-compatible adhesive to at least part of the medical device prior to the step of applying at least part of the liquid tissue product to at least part of the medical device.

The present disclosure includes disclosure of a coated medical device, wherein the medical device comprises a stent, and wherein the coated medical device is configured so that a thrombus will not adhere thereto.

The present disclosure includes disclosure of a method of treating a patient, comprising the step of inserting at least part of a coated medical device into a bloodstream of a patient, the coated medical device produced by harvesting a tissue from a mammal, the tissue selected from the group consisting of pulmonary ligament, mediastinal pleura, parietal pleura, and visceral pleura, to obtain a harvested tissue; freezing the harvested tissue to obtain frozen tissue; mechanically processing the frozen tissue to obtain a liquid tissue product; and applying at least part of the liquid tissue product to at least part of a medical device so to at least partially coat the medical device with the liquid tissue, to produce a coated medical device.

The present disclosure includes disclosure of a method of treating a patient, wherein the medical device comprises a stent, and wherein the coated medical device is configured so that a thrombus will not adhere thereto.

The present disclosure includes disclosure of a method of treating a patient, wherein the medical device comprises a thrombus diverter, and wherein the coated medical device is configured so that a thrombus will not adhere thereto.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1A shows a block diagram identifying various tissues useful to coat devices, according to an exemplary embodiment of the present disclosure;

FIG. 1B shows a block diagram of various medical devices which can be coated using biological material, according to an exemplary embodiment of the present disclosure;

FIG. 2 shows a block diagram of steps of a method to generate a coated medical device, with the various tissues or stages thereof identified in connection with each step, according to an exemplary embodiment of the present disclosure;

FIG. 3A shows a side cut-away view of part of a luminal organ with a traditional device positioned therein and a thrombus formed within the traditional device, according to an exemplary embodiment of the present disclosure;

FIG. 3B shows a side cut-away view of part of a luminal organ with a coated device positioned therein and no thrombus formed therein, according to an exemplary embodiment of the present disclosure; and

FIGS. 4A and 4B show cut-away (cross-sectional views) of coated devices, according to exemplary embodiments of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure contains disclosure of novel methods and uses for harvesting and processing certain mammalian tissue for use in connection with various medical applications. The present disclosure also includes disclosure of various methods of coating medical devices with various tissues referenced herein. Various types of tissue within the scope of the present disclosure are described as follows.

The mammalian pulmonary ligament (also referred to as “pulmonary region tissue”), the mammalian visceral pleura (also referred to as “pulmonary region tissue”), the mammalian parietal pleura, and the mammalian mediastinal pleura (derived from or part of parietal pleura), as referenced in detail below and disclosed within the present application, can be harvested, processed, and used for a number of medical applications previously unknown and not identified in the medical arts. In at least one embodiment of the present disclosure, one or more of the above-referenced tissues is identified, harvested, processed, and ultimately used in connection with mammalian treatment/therapy. Pulmonary ligament 30 (also referred to a pulmonary ligament tissue 30), visceral pleura 556 (also referred to visceral pleura tissue 556, pulmonary pleura 556, or pulmonary pleura tissue 556), parietal pleura 554 (also referred to as parietal pleura tissue 554), and mediastinal pleura 38 (also referred to herein as mediastinal pleura tissue 38) are described in further detail below, whereby one or more of said tissues may be individually or collectively referred to as a tissue 111.

A pleura is a serous membrane that folds back onto itself to form a two-layered membrane structure. Generally, the outer pleura (parietal pleura 554) lines the thoracic cavity, whereas the inner pleura (pulmonary or visceral pleura 556) covers the lungs. The parietal pleura 554 lines the inner surface of the chest wall, covers the superior surface of the diaphragm and encases all of the thoracic viscera (excluding the lungs). Accordingly, the parietal pleura 554 separates the pleural cavity (where the lungs are positioned) from the mediastinum or the “middle” section of the chest cavity.

The parietal pleura 554 is divided into different portions according to its position. For example, the costal pleura is the portion of the parietal pleura 554 that lines the inner surfaces of the ribs and intercostals, the diaphragmatic pleura is that which lines the convex surface of the diaphragm, and the cervical pleura is the portion that rises into the neck and over the apex of the lung. Furthermore, mediastinal pleura 38 is the portion of parietal pleura 554 that defines the mediastinum and encases all of the thoracic viscera except for the lungs, as it runs therebetween.

As the mediastinal pleura 38 separates the right and left lungs, inflation of the lungs causes a corresponding expansion of the mediastinal membrane, thereby resulting in significant friction between the mediastinal pleura 38 and the lungs' surfaces during breathing. While the mediastinal pleura 38 is relatively thin, it nevertheless exhibits substantial integrity and elasticity to accommodate the lungs' expansion and tolerate the friction imposed thereby. The significant elasticity of the mediastinal tissue is indicative of its composition, which consists of multiple fiber-sheet layers having an abundance of elastin fibers in addition to the collagen typically present in connective tissue.

The visceral pleura 556 covers the lungs and extends to the hilum where it becomes continuous with the parietal pleura 554. As the anterior and posterior pleura extend below the pulmonary veins, the two layers of pleura come together to form the inferior pulmonary ligament. Hence, the pulmonary ligament is a double layer of pleura that drapes caudally from the lung root and loosely tethers the medial aspect of the lower lobe of the lung to the mediastinum. However, and importantly, the pulmonary ligament 30 does not functionally behave the same as two layers of pleura, as the non-isotropy of pulmonary ligament 30 tissue is notably different than just two layers of pleura. Furthermore, the degree of collagen within pulmonary ligament 30 is also different than in two layers of pleura, and the function of pulmonary ligament 30 is also different, as pulmonary ligament tissue 30 resists load in one direction. The pulmonary ligament 30 tethers the lung and has substantial elasticity (over 200% extension, which may be a lateral extension) to expand with each inflation of the lung. Similar to the mediastinal pleura 38 previously discussed, the significant elasticity of the pulmonary ligament tissue 30 stems from its high elastin content.

For various pulmonary ligament 30 and/or visceral pleura 556 samples, a predominant proportion of the collagen fibers in the tissue are oriented generally in a first direction, with that direction extending substantially parallel to the median (or midsagittal) plane of the animal from which the tissue was harvested. For example, and in at least one embodiment, at least 75% of collagen fibers within the harvested pulmonary ligament 30 and/or visceral pleura 556 tissue are oriented in a first direction. In at least another embodiment, at least 60% of collagen fibers within the harvested pulmonary ligament 30 and/or visceral pleura 556 tissue are oriented in a first direction. Furthermore, and in various pulmonary ligament 30 and/or visceral pleura 556 samples, said tissues include elastin fibers that extend in a direction transverse to that of the predominating collagen fibers contained therein. However, with respect to mediastinal pleura 38, collagen fibers do not orient in any particular direction and, hence, the mediastinal pleura 38 is mechanically more isotropic than the pulmonary ligament 30 and/or visceral pleura 556.

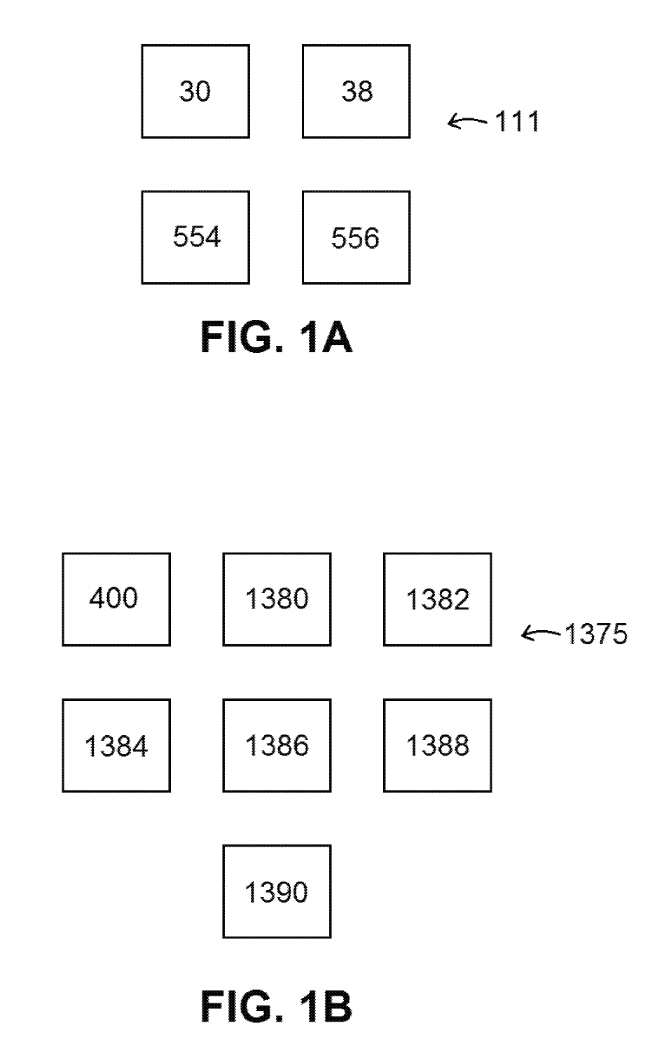

The various types of tissue 111 referenced herein, as noted above, are shown in block format in FIG. 1A.

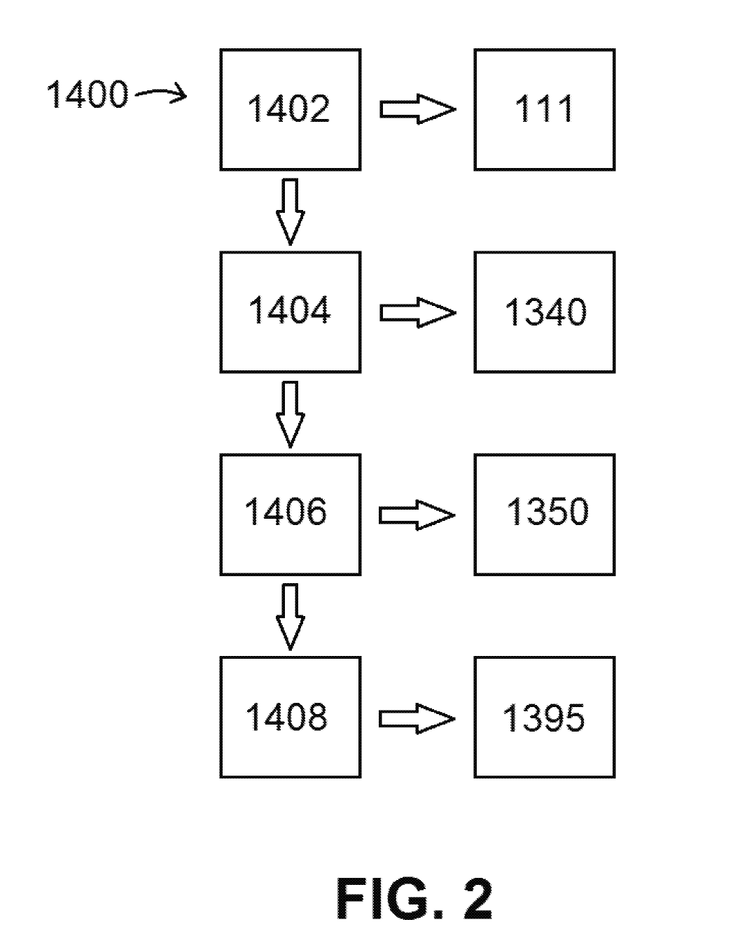

The present disclosure includes disclosure of processing tissue 111 as follows. For example, and as shown by the steps within method 1400 shown in block format in FIG. 2. As shown in FIG. 2, method 1400 comprises the step of harvesting tissue 111 from a mammal (referred to herein as harvesting step 1402), and subsequently freezing the harvested tissue 111, such as by way of freezing in liquid nitrogen, using a low-temperature freezer, or by way of placing harvested tissue within another chemical or device known or developed in the art to reduce the temperature of the same below 32° F., such as below 20° F., 10° F., 0° F., −10° F., or lower (referred to herein as freezing step 1404) to obtain frozen tissue 1340. Once the harvested tissue 111 has been frozen for a desired period of time, such as whereby the harvested tissue 111 has the same or similar temperature to the freezing conditions or such as whereby the harvested tissue 111 is placed in the freezing conditions for a desired amount of time), said tissue can then be mechanically processed (blended, grinded, diced, pulverized, etc.) to form a liquid tissue product 1350 (referred to as mechanical processing step 1406). Liquid tissue product 1350, as referenced herein, can be considered as more of a paste in various embodiments, as liquid tissue product 1350 is the result of mechanically processing frozen tissue 1340, which is frozen harvested tissue 111.

The liquid tissue product 1350, formed as referenced above, can then be applied to one or more medical devices 1375 (as referenced in FIG. 1B in block format), such as stents 1380, stent valves 400, endoprostheses 1382, catheters 1384, thrombosis diverters 1386, leads 1388, cannulas 1390, and/or other medical devices 1375 known or developed in the art that are configured to be temporarily or permanently positioned within a mammalian luminal organ 850 (such as a blood vessel, for example), of a mammalian patient, with such a step referred to herein as a device coating step 1408, to generate a coated medical device 1395.

Device coating step 1408 may optionally include the step of partially or fully coating a medical device 1375 with a biologically-compatible adhesive 1450, such as a biological glue, platelets, and/or another adhesive known or developed in the medical arts useful to adhere to a medical device 1375 and/or to combine with a liquid tissue product 1350 prior to applying liquid tissue product 1350 to medical device 1375 to generate coated medical device 1395. Device coating step 1408 may then optionally include the step of combining adhesive 1450 with liquid tissue product 1350, and then combining that combination to medical device 1375. Adhesive 1450, in various embodiments, helps liquid tissue product 1350 become “sticky” so that it can adhere to medical device 1375. In the same or other embodiments, adhesive 1450 is configured to or capable of adhering to medical device 1375 and to liquid tissue product 1350, so to ensure adhesion of liquid tissue product 1350 to medical device 1375.

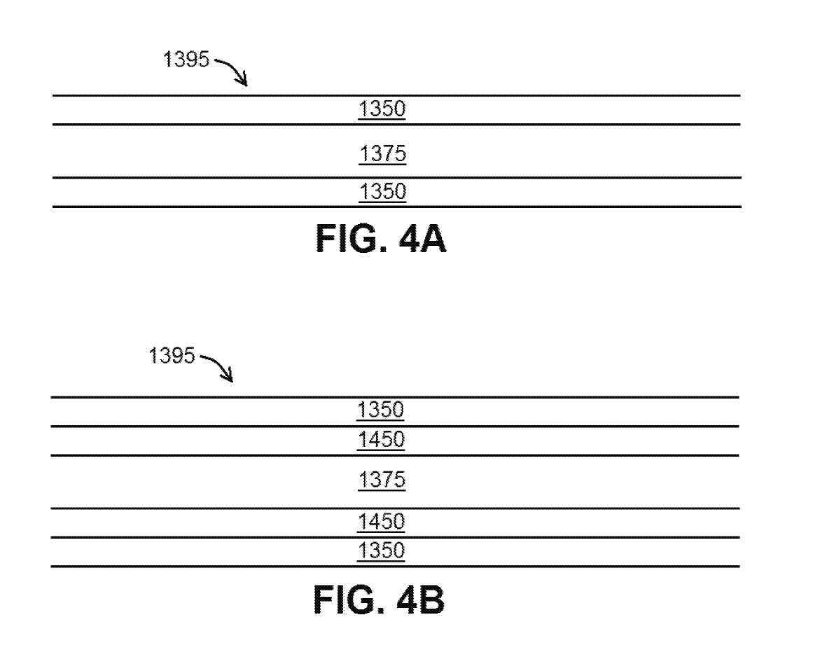

FIG. 4A shows a cut-away (cross-sectional) view of a portion of a coated medical device 1395, whereby medical device 1375 is coated with liquid tissue product 1350. Such an embodiment of a coated medical device 1395 refers to one generated by a) applying liquid tissue product 1350 to medical device 1375, or b) combining liquid tissue product 1350 with an adhesive 1450 and applying that combination to medical device 1375. FIG. 4A shows a cut-away (cross-sectional) view of a portion of a coated medical device 1395, whereby medical device 1375 is coated with an adhesive 1450 prior to being coated with a liquid tissue product 1350 of the present disclosure.

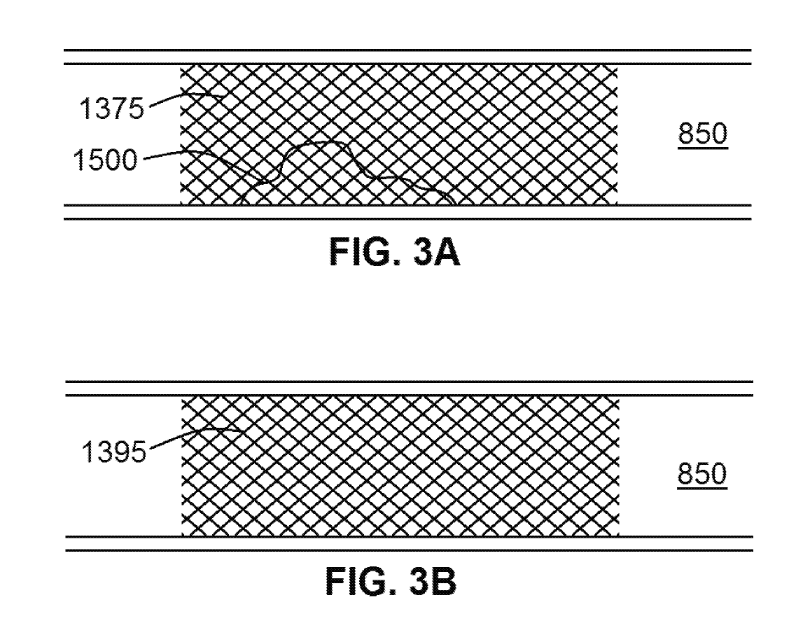

Liquid tissue product 1350, formed using a processed tissue 111 of the present disclosure, has been identified as having previously unknown thrombosis-resistant properties. Said properties were confirmed, in connection with studies performed relating to the present disclosure, by preparing a coated medical device 1395 configured as a stent and identifying that when the coated medical device 1395 was positioned in an environment such as positioning a traditional stent 1380 within a blood vessel (an exemplary mammalian luminal organ 850), blood particulates (blood cells and/or other matter within a bloodstream) did not adhere to the coated medical device 1395, as shown in FIG. 3B. Conversely, and over time, a traditional stent 1380 would ultimately collect particulate matter from the blood stream, forming a localized thrombus 1500 (as shown in FIG. 3A), which if detached could block a blood vessel, such as in the periphery and/or the brain, causing serious injury or even death, such as by way of stroke.

In view of the foregoing, coated medical devices 1395 can be used in connection with various medical procedures to treat various medical conditions.

The present disclosure therefore includes disclosure of producing coated medical devices 1395 by performing method 1400 steps referenced herein. Method 1400 may include other steps, and may start at any step noted herein, such as where frozen tissue is obtained and processed by performing mechanical processing step 1406, for example.

While various embodiments of biological material-coated devices and methods for producing and using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A method to produce a coated medical device, the method comprising the steps of:
harvesting a tissue from a mammal, the tissue selected from the group consisting of pulmonary ligament, mediastinal pleura, parietal pleura, and visceral pleura, to obtain a harvested tissue;
freezing the harvested tissue to obtain frozen tissue;
mechanically processing the frozen tissue to obtain a liquid tissue product; and
applying at least part of the liquid tissue product to at least part of a medical device so to at least partially coat the medical device with the liquid tissue, to produce a coated medical device.

2. The method of claim 1, wherein the tissue comprises pulmonary ligament, and wherein the step of mechanically processing the frozen tissue is performed by blending or grinding the frozen tissue.

3. The method of claim 1, wherein the tissue comprises mediastinal pleura, and wherein the step of mechanically processing the frozen tissue is performed by blending or grinding the frozen tissue.

4. The method of claim 1, wherein the tissue comprises parietal pleura, and wherein the step of mechanically processing the frozen tissue is performed by blending or grinding the frozen tissue.

5. The method of claim 1, wherein the tissue comprises visceral pleura, and wherein the step of mechanically processing the frozen tissue is performed by blending or grinding the frozen tissue.

6. The method of claim 1, wherein the step of applying is performed to apply at least part of the liquid tissue product to at least part of the medical device selected from the group consisting of a stent, a stent valve, an endoprosthesis, a catheter, a thrombus diverter, a lead, and a cannula.

7. The method of claim 1, further comprising the step of:
combining a biologically-compatible adhesive with the liquid tissue product prior to the step of applying at least part of the liquid tissue product to at least part of the medical device.

8. The method of claim 1, further comprising the step of:
applying a biologically-compatible adhesive to at least part of the medical device prior to the step of applying at least part of the liquid tissue product to at least part of the medical device.

9. A coated medical device, generated by:
harvesting a tissue from a mammal, the tissue selected from the group consisting of pulmonary ligament, mediastinal pleura, parietal pleura, and visceral pleura, to obtain a harvested tissue;
freezing the harvested tissue to obtain frozen tissue;
mechanically processing the frozen tissue to obtain a liquid tissue product; and
applying at least part of the liquid tissue product to at least part of a medical device so to at least partially coat the medical device with the liquid tissue, to produce a coated medical device.

10. The coated medical device of claim 9, wherein the tissue comprises pulmonary ligament, and wherein the step of mechanically processing the frozen tissue is performed by blending or grinding the frozen tissue.

11. The coated medical device of claim 9, wherein the tissue comprises mediastinal pleura, and wherein the step of mechanically processing the frozen tissue is performed by blending or grinding the frozen tissue.

12. The coated medical device of claim 9, wherein the tissue comprises parietal pleura, and wherein the step of mechanically processing the frozen tissue is performed by blending or grinding the frozen tissue.

13. The coated medical device of claim 9, wherein the tissue comprises visceral pleura, and wherein the step of mechanically processing the frozen tissue is performed by blending or grinding the frozen tissue.

14. The coated medical device of claim 9, wherein the step of applying is performed to apply at least part of the liquid tissue product to at least part of the medical device selected from the group consisting of a stent, a stent valve, an endoprosthesis, a catheter, a thrombus diverter, a lead, and a cannula.

15. The coated medical device of claim 9, further generated by combining a biologically-compatible adhesive with the liquid tissue product prior to the step of applying at least part of the liquid tissue product to at least part of the medical device.

16. The coated medical device of claim 9, further generated by applying a biologically-compatible adhesive to at least part of the medical device prior to the step of applying at least part of the liquid tissue product to at least part of the medical device.

17. The coated medical device of claim 9, wherein the medical device comprises a stent, and wherein the coated medical device is configured so that a thrombus will not adhere thereto.

18. A method of treating a patient, comprising the step of:
inserting at least part of a coated medical device into a bloodstream of a patient, the coated medical device produced by:
harvesting a tissue from a mammal, the tissue selected from the group consisting of pulmonary ligament, mediastinal pleura, parietal pleura, and visceral pleura, to obtain a harvested tissue;
freezing the harvested tissue to obtain frozen tissue;
mechanically processing the frozen tissue to obtain a liquid tissue product; and
applying at least part of the liquid tissue product to at least part of a medical device so to at least partially coat the medical device with the liquid tissue, to produce a coated medical device.

19. The method of claim 18, wherein the medical device comprises a stent, and wherein the coated medical device is configured so that a thrombus will not adhere thereto.

20. The method of claim 18, wherein the medical device comprises a thrombus diverter, and wherein the coated medical device is configured so that a thrombus will not adhere thereto.
